(19) United States
(12) Patent Application Publication
(10) Pub. No.: US 2026/0053565 A1
(43) Pub. Date: Feb. 26, 2026

(54) DYNAMIC VISUALIZATION OF EXPECTED ABLATION ZONE

(71) Applicant: Neuwave Medical, Inc., Madison, WI (US)

(72) Inventor: Matthew SCHANING, Cambridge, WI (US)

(73) Assignee: Neuwave Medical, Inc., (US)

(21) Appl. No.: 18/811,266

(22) Filed: Aug. 21, 2024

(51) Int. Cl. A61B 34/10; A61B 18/00; A61B 34/00

(52) U.S. Cl. CPC A61B 34/10; A61B 18/00; A61B 34/25; A61B 2018/00577; A61B 2018/00702; A61B 2018/00761; A61B 2018/00791; A61B 2034/104

(57) ABSTRACT

A system is disclosed including an ablation probe that includes an antenna, a display, and a controller in communication with the ablation probe and the display. The controller comprises a memory and is operable to receive first and second inputs indicative of an amount of time and a power level, respectively, to energize the ablation probe, retrieve, from the memory, a predicted ablation zone corresponding to the first and second inputs, display, on the display, an image representative of the ablation probe, and overlay, on the image, the predicted ablation zone, energize the ablation probe at the power level, and dynamically overlay, on the image, a progressive ablation zone based on the ablation probe being energized, wherein a size of the progressive ablation zone is retrieved from the memory based on the amount of time the ablation probe has been energized at the power level.

### BACKGROUND

Ablation is an important therapeutic strategy for treating certain tissues, such as benign and malignant tumors, cardiac arrhythmias, cardiac dysrhythmias, and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

When performing an ablation procedure, it could be helpful to visualize the expected ablation treatment area during a procedure or “mid-procedure”. Accordingly, there is a need for improved systems and methods that enable a physician to visualize the expected ablation treatment area mid-procedure.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system that may be used in accordance with the principles of the present disclosure.

FIG. 2 is a schematic diagram of an example energy delivery device that may be used in accordance with the principles of the present disclosure.

FIG. 3 illustrates a graphical user interface (GUI) of the energy delivery system of FIG. 1 displaying an image representative of the energy delivery device of FIG. 2 and a predicted ablation zone overlaid thereon according to a set time and power, according to at least one aspect of the present disclosure.

FIG. 4 is a graph providing an expected longitudinal length of an ablation zone over time for various power levels, according to at least one aspect of the present disclosure.

FIG. 5 is a look-up table providing an expected longitudinal length of an ablation zone over time for a power level, according to at least one aspect of the present disclosure.

FIG. 6 illustrates the GUI of FIG. 3 further including a first state of a progressive ablation zone after a first amount of elapsed time, according to at least one aspect of the present disclosure.

FIG. 7 illustrates the GUI of FIG. 6 where the progressive ablation zone has been transitioned from the first state to a second state after a second amount of elapsed time, according to at least one aspect of the present disclosure.

FIG. 8 illustrates a graphical user interface (GUI) of the energy delivery system of FIG. 1 displaying a three-dimensional image representative of a plurality of energy delivery devices of FIG. 2, a lesion, and an anatomical structure, with a predicted ablation zone and a progressive ablation zone overlaid on one of the plurality of energy delivery devices, according to at least one aspect of the present disclosure.

### DETAILED DESCRIPTION

The present disclosure is related to systems and methods for delivering energy to tissue for ablation operation and, more particularly, to systems and methods for graphically visualizing an expected ablation treatment area mid-procedure.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

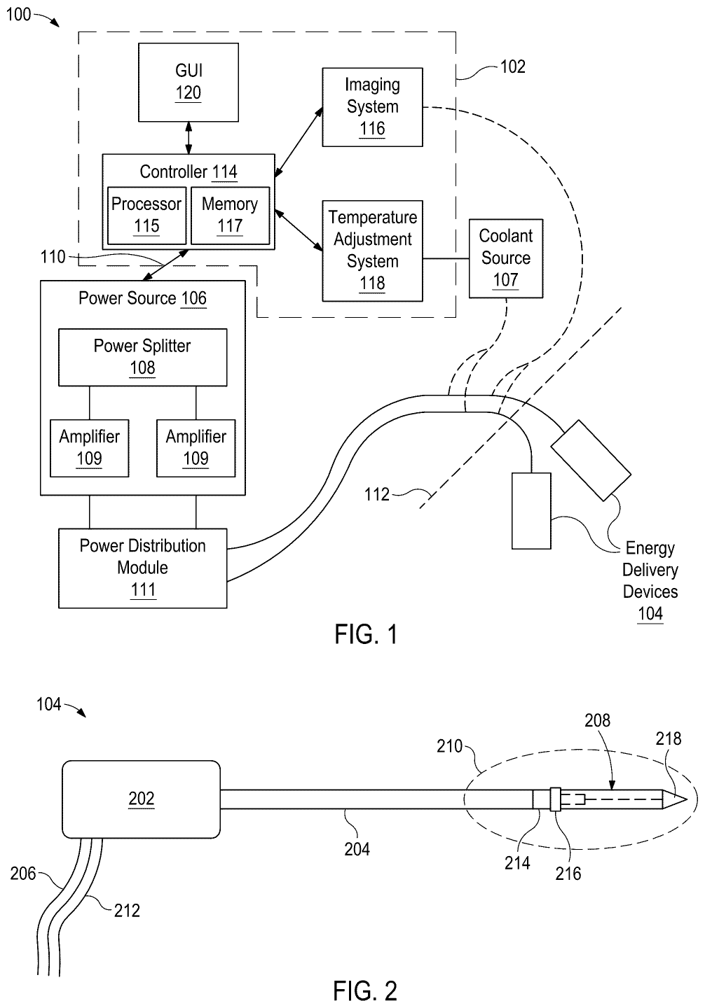

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes) An example ablation probe 104 that can be used with the energy delivery system 100 is described in U.S. Patent Application Publication No. 2021/0282851, entitled “ENERGY DELIVERY SYSTEMS AND USES THEREOF”, which published on Sep. 16, 2021, the disclosure of which is hereby incorporated by reference in its entirety.

The system 100 further includes a power source or generator 106 communicably coupled to the control system 102 and the ablation probes 104 to direct, control, and deliver (provide) electrical power thereto. In some applications, the power source 106 may include a power splitter 108 that receives power from an external power source (e.g. a wall outlet) and directs power to one or more amplifiers 109 (two shown), which amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which provides strain relief to the cabling extending from the amplifiers 109 to the ablation probes 104. The power distribution module 111 may be coupled to a structure in the operating room, such as a surgical bed, and may house connection hardware of the probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

FIG. 2 is a schematic diagram of an example probe 104 that may be used in accordance with the principles of the present disclosure. As indicated above, the ablation probe 104 may be configured to deliver (emit) energy (e.g., microwave energy, radiofrequency energy, radiation energy) to a target tissue region. As illustrated, the ablation probe 104 includes a handle housing 202 and an elongate shaft or probe cannula 204 extending distally from the handle 202.

A cable or cable assembly 206 may be operatively coupled to the handle 202 and configured to convey electrical power thereto. The cable assembly 206 may extend from the power distribution module 111 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines). The material of the antenna 208 is durable and provides a high dielectric constant. In some applications, the material of the antenna 208 is zirconium and/or a functional equivalent of zirconium. In at least one application, the ablation probe 104 includes two or more separate antennae 208 attached to the same or different power supplies.

In some applications, a cooling tube 212 is operatively coupled and configured to convey a cooling fluid or “coolant” to the handle 202. The handle 202 may be configured to control conveyance of the cooling fluid into and out of the probe cannula 204 to help regulate a temperature of the antenna 208 and the ablation zone 210. Example cooling fluids include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula at or near the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO₂) that freezes the interface between the stick region 214 and the adjacent tissue, thereby sticking (maintaining, locking, etc.) the antenna 208 in place during operation. The coolant may be provided to the stick region 214 via the cooling tube 212 from the coolant source 107, for example. Once a pre-determined low temperature is reached at the stick region 214, contact with adjacent tissue causes the tissue to adhere to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that upon contact with tissue induces adherence of the tissue onto the stick region 214. Example materials for the stick region 214 include, but are not limited to, a metal.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. Accordingly, the plug region 216 separates interior portions of the ablation probe 104 to prevent cooling or heating of a portion or portions of the probe 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fiberglass, cork, or any combination thereof.

In some applications, the ablation probe 104 may further include a sharp stylet tip or “stylet” 218 positioned at the distal end of the antenna 208 and otherwise forming the distal end of the ablation probe 104. When included, the stylet 218 is designed to facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

In some applications, the ablation probe 104 may have a coaxial transmission line positioned within the antenna 208, and a coaxial transmission line connecting with the antenna 208. In other embodiments, the ablation probe 104 may comprise a triaxial microwave probe with optimized tuning capabilities. The ablation probe 104 may be the same as or similar to any of the energy delivery devices described in U.S. Pat. No. 11,638,607, entitled “ENERGY DELIVERY SYSTEMS AND USES THEREOF”, which issued on May 2, 2023, the contents of which are hereby incorporated by reference in their entirety herein.

Referring again to FIG. 1, the control system 102 is configured to monitor, control, and provide feedback concerning operation of the system 100. As illustrated, the control system 102 includes at least a controller 114, an imaging system 116, and a temperature adjustment system 118. The control system 102 may further include a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The control system 102 further includes the imaging system 116, which is in communication with the controller 114 and comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the system 100 provides software configured for use with imaging equipment of the imaging system 116, such as CT, MRI, and ultrasound, and generate two-dimensional (2D) and or three-dimensional (3D) images viewable by a user on the GUI 120. In some embodiments, the imaging equipment software allows a user to make predictions based upon known thermodynamic and electrical properties of tissue, vasculature, and location of the antenna(s) 208 (FIG. 2). In some embodiments, the imaging software allows the generation of a 2D or 3D map of the location of a tissue region (e.g., tumor, arrhythmia), location of the antenna(s) 208, and to generate a predicted map of the ablation zone 210 (FIG. 2).

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power source 106 may be configured to supply the energy required to operate the system 100. The power source 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power source 106 supplies microwave energy to the ablation probes 104 for purposes of tissue ablation. More specifically, power may be supplied to the ablation probes 104, but the microwave energy is generated in a microwave generator and sent to the antenna 208. In some applications, the power source 106 may include one or more energy generators configured to provide as much as 140-150 watts of microwave power of a frequency of from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The temperature adjustment system 118 may be configured to use coolant systems (like the coolant source 107) and cooling fluids to help reduce undesired heating within and along the ablation probes 104. In particular, the temperature adjustment system 118 may include the coolant source 107 and the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface. For instance, in some embodiments, a user provides an input to an input interface, such as the GUI 120. Based on the user input, the temperature adjustment system 118 can control the coolant source 107 to provide coolant to the stick region 214, thereby adhering the stick region 214 to the adjacent tissue.

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a first temperature sensor, a second temperature sensor, and a third temperature sensor. The first temperature sensor is placed at, or slightly proximal to, the antenna 208 to provide real-temperature measurements of the tissue being heated by the antenna 208. The second temperature sensor is placed at, or adjacent to, the stick region 214 to provide real-time temperature measurements of the tissue that is being cooled, and thus adhered to, the stick region 214. The third temperature sensor is proximal to the first and second temperature sensors along the cannula 204, such as at the point of entry into the skin, to provide real-time measurements of the patent's skin. The control system 102 can receive the temperature measurements from the first, second, and third sensors to control the coolant systems and cooling fluids from the temperature adjustment system 118 to the stick region 214 and/or other cooling systems of the energy delivery device 104, as will be described in more detail below.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the controller 114 to provide real-time temperature information to a user and display such measurements on the GUI 120. In at least one embodiment, based on the temperature data obtained by the temperature adjustment system 118, the controller 114 may be configured to autonomously adjust operation of the system 100 appropriately.

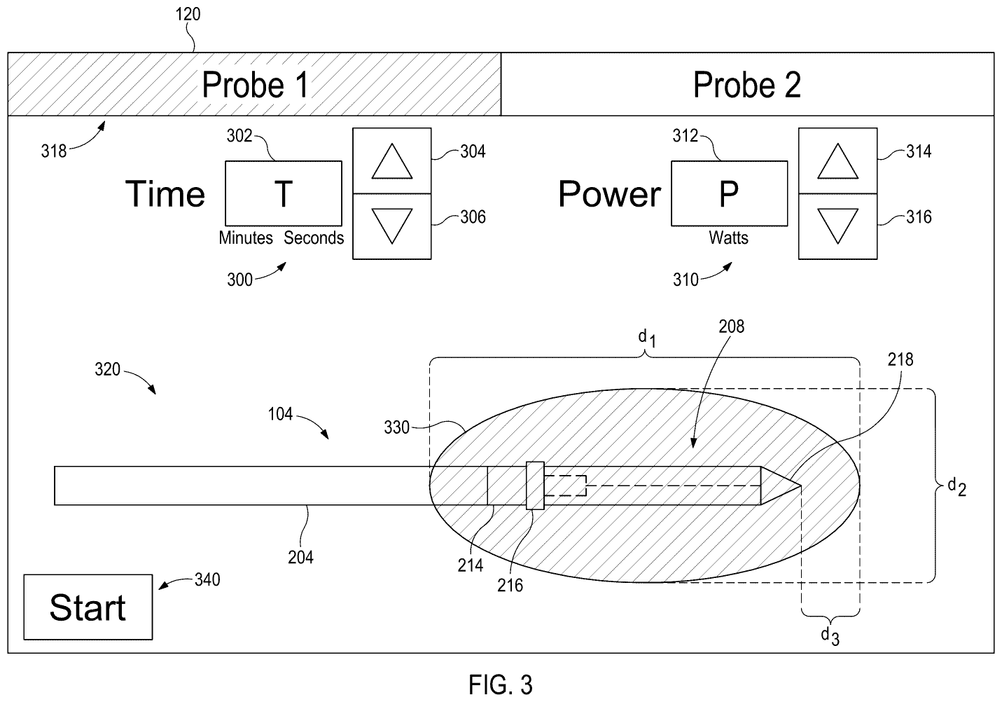

Referring now to FIG. 3, with continued reference to FIG. 1, illustrated is a schematic illustration of one example of the GUI 120, according to the principles of the present disclosure. The controller 114 may allow a user to choose, among other things, a power level and a duration of treatment (amount of time) at which to energize each of the ablation probes 104. For instance, the controller 114 may display, on the GUI 120, a time widget 300 and a power widget 310 for a selected one of the ablation probes 104 of the system 100 (“Probe 1”). The time widget 300 may include a display portion 302 for displaying the set amount of time T to energize the selected ablation probe 104. The time widget 300 may further include interactive controls 304, 306 for adjusting the set duration of treatment for the selected ablation probe 104. A user may provide an input to (interact with) the first control 304 to increase the time duration of the treatment, or the second control 306 to decrease the time duration of treatment.

Similarly, the power widget 310 may include a display portion 312 for displaying the set power P to energize the selected ablation probe 104. The power widget 310 may further include interactive controls 314, 316 for adjusting the set power level for the selected ablation probe 104. A user may provide an input to (interact with) the first control 314 to increase the power output (level) of the treatment, or the second control 316 to decrease the power output (level) of the treatment.

The controller 114 may also display, on the GUI 120, an image 320 representative of at least a portion of the ablation probe 104 selected to be energized. As shown in FIG. 3, the image 320 may include at least a portion of the cannula 204 and the antenna 208. Alternatively, the image 320 may omit the cannula 204, or the image 320 may include the entire ablation probe 104 (i.e., the handle housing 202, the cannula 204, the antenna 208, etc.). The image 320 may be a two-dimensional (2D) or three-dimensional (3D) image and may be stored in the memory 117.

Based on receiving the time duration of treatment and power level, via the time and power widgets 300, 310, respectively, the controller 114 may overlay, on the image 320, a predicted ablation zone 330 that is expected to be produced for the selected amount of time and power level. The memory 117 may have stored thereon data that correlates set times and power levels to expected (predicted) dimensions for the ablation zone 330. The data may include ex-vivo data, in-vivo data, or clinical data, or combinations thereof. As illustrated, and based on the data correlated to the set time and power level, the predicted ablation zone 330 may be displayed with an expected longitudinal length d₁ an expected lateral width d₂, and an expected distance d₃ between a distal end of the stylet 218 and a distal-most end of the predicted ablation zone 330. The resulting shape of the predicted ablation zone 330 may form an ellipse, an ellipsoid, or a circle.

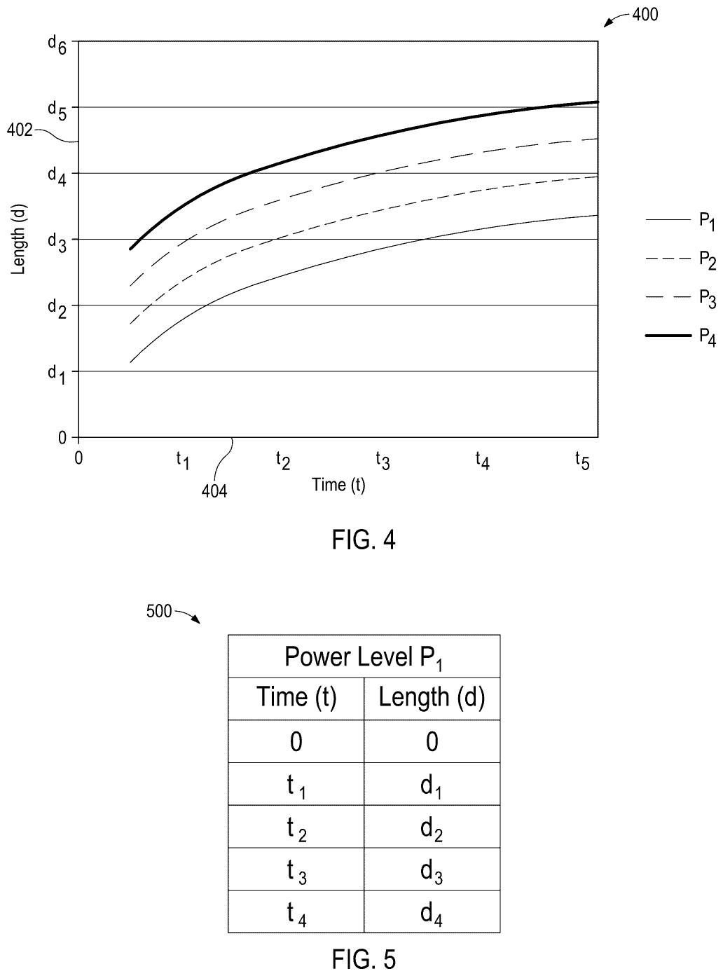

Referring to FIG. 4, for example, the memory 117 may store data related to a graph 400 that provides an expected longitudinal length 402 (d₁ of FIG. 3) of the predicted ablation zone 330 over time 404 for various power levels P₁-P₄. With reference to FIG. 5, the memory 117 may further store data related to one or more look-up tables 500 (only one shown) that correlates set times to an expected longitudinal length (d₁ of FIG. 3) of the predicted ablation zone 330 over time for specific power levels. It should be understood that the memory 117 may store individual graphs 400 and/or look-up tables 500 for each dimension of interest (e.g. d₁- d₃; see FIG. 3) for the predicted ablation zone 330 at various power levels, as will be described in more detail below.

Referring again to FIG. 3, the controller 114 may interrogate the data (e.g., the graphs 400 (FIG. 4) and/or tables 500 (FIG. 5)) stored in the memory 117 to determine dimensions for the predicted ablation zone 330 for the set time and power. For example, the controller 114 may interrogate the data to determine the expected longitudinal length d₁ of the predicted ablation zone 330, the expected lateral width d₂ of the predicted ablation zone 330, and/or the expected distance d₃ defined between a distal end of the stylet 218 and a distal-most end of the predicted ablation zone 330. The controller 114 may overlay the predicted ablation zone 330 according to the determined dimensions d₁-d₃ retrieved from the memory 117, which may provide a user with an indication of an ablation zone that is expected to be produced for the set time/power.

The controller 114 may further overlay the determined dimensions d₁-d₃ on the GUI 120 for the user's information. In some embodiments, the user may interact with the controls 304, 306, 314, 316 to selectively adjust the set time/power, and the controller 114 may dynamically adjust the overlaid predicted ablation zone 330 and overlaid dimensions d₁-d₃ according to the updated set time/power.

Once the user is satisfied with the predicted ablation zone 330 to be produced for the set time and power level, the user may choose to energize the selected ablation probe 104. For example, the controller 114 may proceed with energizing the ablation probe 104 at the set power level based on a user providing an input to (interacting with) a power widget 340 displayed on the GUI 120. Alternatively, the controller 114 may proceed with energizing the ablation probe 104 at the set power level based on a user providing an input to a switch, such as a foot pedal or switch, or the like.

According to embodiments of the present disclosure, once the ablation probe 104 is energized at the set power level, the controller 114 may then be configured to interrogate, continuously or periodically, the data stored in the memory 117 (e.g., the graphs 400 of FIG. 4, the tables 500 of FIG. 5, etc.) to determine dimensions for the predicted ablation zone 330 expected to be produced at the set power over time. Based on the determined dimensional information, the controller 114 may then be programmed to dynamically overlay on the image 320 a progressive ablation zone corresponding to an elapsed amount of time from the power widget 340 being interacted with.

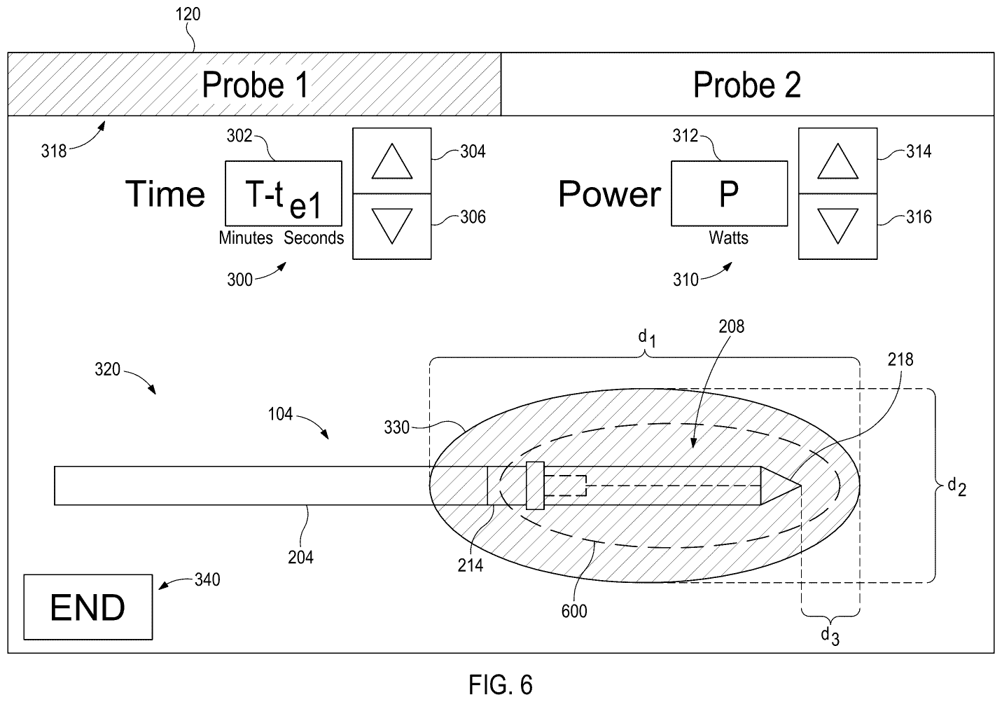

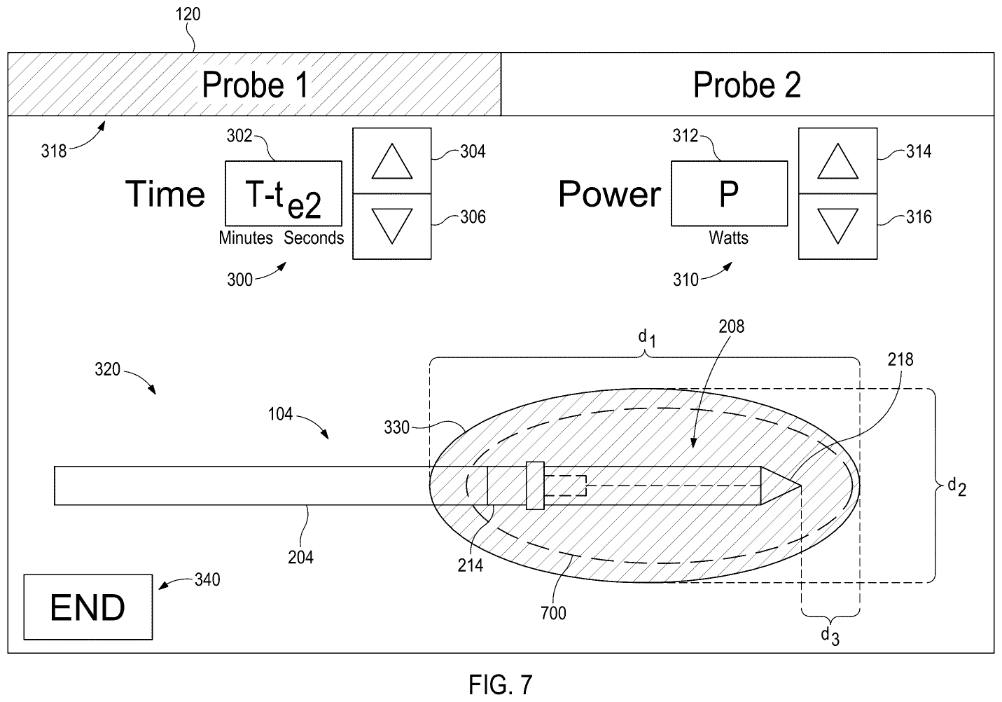

Referring to FIG. 6, for example, after a first amount of time has elapsed since the ablation probe 104 has been energized (tₑ₁), the controller 114 may determine, via the data stored in the memory 117, that the ablation zone is expected to have a first longitudinal length, a first lateral width, and a first distance defined between the stylet 218 and a distal-most end of the ablation zone. According to the determined dimensions, the controller 114 may further overlay, on the image 320, a first state of a progressive ablation zone 600, which is smaller than the predicted ablation zone 330. The progressive ablation zone enables a user to visualize a real-time size of the ablation zone produced by the probe 104. As will be described in more detail below, the controller 114 may transition the progressive ablation zone between a plurality of states with each state representative of the size of the real-time size of the ablation zone at a particular time. For example, the controller 114 may increase the size of the progressive ablation zone, like a balloon, for example, from the first state of the progressive ablation zone 600 (FIG. 6) to a second state of the progressive ablation zone 700 (FIG. 7), or the controller 114 may update the progressive ablation zone by removing the first state of the progressive ablation zone 600 (FIG. 6) and replacing it with a larger-dimension second state of the progressive ablation zone 700 (FIG. 7). The controller 114 may further decrement the set time T on the display 302 of the time widget 300 according to the elapsed amount of time tₑ₁. Accordingly, a user can view on the GUI 120 the amount of time remaining in the procedure (T−tₑ₁), via the display 302, and how much the ablation zone is expected to have grown over the elapsed amount of time tₑ₁ via the first state of the progressive ablation zone 600.

Referring to FIG. 7, after a second amount of time has elapsed since the ablation probe 104 has been energized (tₑ₂), where tₑ₂>tₑ₁, the controller 114 may determine, via the data stored in the memory 117, that the ablation zone is expected to have a second longitudinal length greater than the first longitudinal length, a second lateral width greater than the first lateral width, and a second distance defined between the stylet 218 and a distal-most end of the ablation zone greater than the first distance. According to the determined dimensions, the controller 114 may remove from the image 320 the overlaid first state of the progressive ablation zone 600 and overlay on the image 320 a second state of the progressive ablation zone 700, which is larger than the first state of the progressive ablation zone 600, but smaller than the predicted ablation zone 330. Alternatively, as referenced above, the controller 114 may expand the progressive ablation zone from the first state 600 (FIG. 6) to the second state (FIG. 7) like a balloon. The controller 114 may further decrement the set time T on the display 302 of the time widget 300 according to the elapsed amount of time tₑ₂. Accordingly, a user can view on the GUI 120 an amount of time remaining in the procedure (T−tₑ₂) via the display 302, and how much the ablation zone 330 is expected to have grown over the elapsed amount of time tₑ₂, via the second state of the progressive ablation zone 700.

The controller 114 may repeat the foregoing interrogation, removal of the previous state of the progressive ablation zone and corresponding dimensions d₁-d₃, and overlaying of a new state of the progressive ablation zone and corresponding dimensions d₁-d₃ over the entirety of the set amount of time for the ablation procedure, either continuously or periodically, to allow the user to view the expected real-time size of the ablation zone relative to the predicted final size of the ablation zone. As the controller 114 selectively removes and overlays the progressive ablation zones 600, 700, a user may choose to end the ablation procedure prior to the set amount of time T elapsing. For instance, based on the size and extent of the state of the progressive ablation zone depicted on the display 302, a user may decide that a sufficient amount of area is expected to have been ablated prior to the set amount of time T elapsing. Accordingly, at that point the user may stop operation of the selected ablation device 140. In some embodiments, for example, the user may provide an input to (interact with) the power widget 340, causing the controller 114 to cease providing energy to the selected ablation device 140.

As referenced elsewhere herein, localized temperature may be monitored at several points along the probe 104 via one or more temperature sensors (e.g., thermocouples). The controller 114 may receive these sensed temperatures and adjust the state of the progressive ablation zone based thereon. For instance, referring again to FIG. 6, after the first amount of time tₑ₁ has elapsed with the probe energized at power P, the controller 114 may expect to sense a temperature Tₑₓₚ at one of the temperature sensors. The expected temperatures for given times and power may be stored in the memory 117, for example. However, the controller 114 may actually sense a temperature T_{act} that is different (less than or greater than) the expected temperature Tₑₓₚ after the first amount of time tₑ₁ at power P. Based on the difference between the actual temperature T_{act} and the expected temperature Tₑₓₚ, the controller 114 may adjust the state of the progressive ablation zone. For example, the controller 114 may increase the size of the progressive ablation zone based on the actual temperature T_{act} being greater than the expected temperature Tₑₓₚ or decrease the size of the progressive ablation zone based on the actual temperature T_{act} being less than the expected temperature Tₑₓₚ. Accordingly, the controller 114 is able to adjust the predicted, initial dimensions (e.g. d₁-d₃) for a state of the progressive ablation zone based on real-time data sensed by the temperature sensors.

While the foregoing description and figures are described with respect to one ablation probe (“Probe 1”), it should be understood that a similar arrangement of elements would be provided on the GUI 120 for each other ablation probe 104 associated with the system 100 (e.g. “Probe 2”). For instance, the user may provide an input to (interact with) a selection bar 318 that allows a user to select between each of the probes 104 of the system 100 (Probe 1 or Probe 2), thereby allowing a user to set a desired time and power level for each ablation probe 104, view the predicted ablation zones for each of the probes, and monitor the progressive ablation zones of each probe over time. Accordingly, the display of the system 100 enables a user to dynamically control and monitor a plurality of ablation probes 104 over time.

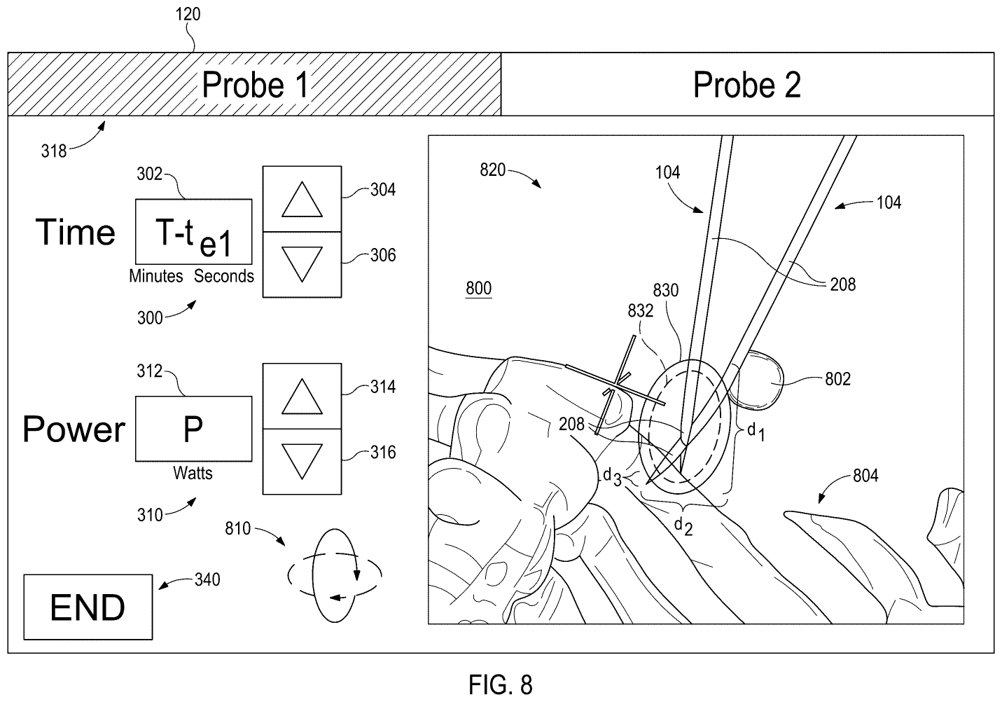

Referring to FIG. 8, in some instances, the controller 114 may receive visualization data from the imaging system 116. The visualization data may include the location or positioning of one or more ablation probes 104 in a patient 800 (detected in real-time or pre-operatively), the location of one or more lesions 802 identified in the patient 800 (detected in real-time or pre-operatively, for example), and/or the location of one or more anatomical structures 804 in the patient 800 (detected in real-time or pre-operatively, for example). Based on the visualization data, the controller 114 may display, on the GUI 120, a three-dimensional (3D) image 820 representative of the ablation probes 104, the one or more lesions 802, and/or the one or more anatomical structure 804.

Similar to above, a user can select between the plurality of probes 104, via the selection bar 318, to set a duration of procedure and power level of the probe 104, via the time and power widgets 300, 310, respectively, view a predicted ablation zone 830 for each of the probes (one shown), and view states of the progressive ablation zones 832 for each of the probes (one shown) over time. Utilizing the visualization data from the imaging system 116 enables a user to position one or more probes 104 relative to a lesion 802, view the predicted ablation zones for each of the probes relative to the lesion 802, and track the progressive ablation zones over time relative to the lesion 802. The user may also interact with the power widget 340 to cease energy delivery to the ablation probe 104 should a user decide that a sufficient amount of the lesion 802 has been expected to have been ablated prior to the amount of time T elapsing. The GUI 120 may also include a rotation widget 850 that a user can provide an input to (interact with) to rotate the 3D image 820 to view the predicted ablation zone 830 and progressive ablation zones 832 for each of the probes over time.

### Embodiments Disclosed Herein Include:

A. A system comprising an ablation probe that includes an antenna, a display, and a controller in communication with the ablation probe and the display. The controller comprises a memory and is operable to receive first and second inputs indicative of an amount of time and a power level, respectively, to energize the ablation probe, retrieve, from the memory, a predicted ablation zone corresponding to the first and second inputs, display, on the display, an image representative of the ablation probe, and overlay, on the image, the predicted ablation zone, energize the ablation probe at the power level, and dynamically overlay, on the image, a progressive ablation zone based on the ablation probe being energized, wherein a size of the progressive ablation zone is retrieved from the memory based on the amount of time the ablation probe has been energized at the power level.

B. A method comprising displaying, on a display, an image representative of an ablation probe, receiving first and second inputs indicative of an amount of time and a power level, respectively, to energize the ablation probe, retrieve, from the memory, a predicted ablation zone corresponding to the first and second inputs, overlaying, on the image, the predicted ablation zone, and dynamically overlaying, on the image, a progressive ablation zone based on the ablation probe being energized, wherein a size of the progressive ablation zone is retrieved from the memory based on the amount of time the ablation probe has been energized at the power level.

C. A non-transitory computer readable medium storing instructions that, when executed by a processor, cause the processor to receive first and second inputs indicative of an amount of time and a power level, respectively, to energize an antenna of an ablation probe, retrieve, from a memory, a predicted ablation zone corresponding to the first and second inputs, display, on a display, an image representative of the ablation probe, and overlay, on the image, the predicted ablation zone, energize the ablation probe at the power level, and dynamically overlay, on the image, a progressive ablation zone based on the ablation probe being energized, wherein a size of the progressive ablation zone is retrieved from the memory based on the amount of time the ablation probe has been energized at the power level.

Each of embodiments A, B, and C may have one or more of the following additional elements in any combination: Element 1: wherein to dynamically overlay a progressive ablation zone, the controller is operable to retrieve, from the memory at a first time, a first state of the ablation zone smaller than the predicted ablation zone, overlay, on the image, the first state of the progressive ablation zone, retrieve, from the memory at a second time, a second state of the ablation zone larger than the first state of the progressive ablation zone and smaller than the predicted ablation zone, wherein the second time is later than the first time, and overlay, on the image, the second state of the progressive ablation zone. Element 2: wherein the controller is further operable to receive a third input prior to the amount of time elapsing and cease energizing the ablation probe based on the third input. Element 3: wherein the controller is further operable to retrieve, from the memory, a dimension associated with the predicted ablation zone and overlay, on the image, the dimension. Element 4: wherein the dimension comprises a longitudinal length of the predicted ablation zone, a lateral width of the predicted ablation zone, or a distance defined between a distal end of the ablation probe and a distal-most end of the predicted ablation zone, or combinations thereof. Element 5: wherein the image comprises a three-dimensional image. Element 6: wherein the display includes a time widget and a power widget, and wherein the controller is further operable to receive the first and second inputs via the time and power widgets, respectively. Element 7: wherein the controller is further operable to receive, from a temperature sensor, a temperature measurement and adjust, on the display, the size of the progressive ablation zone based on the temperature measurement. Element 8: wherein dynamically overlaying a progressive ablation zone comprises retrieving, from the memory at a first time, a first state of the ablation zone smaller than the predicted ablation zone, overlaying, on the image, the first state of the progressive ablation zone, retrieving, from the memory at a second time, a second state of the ablation zone larger than the first state of the progressive ablation zone and smaller than the predicted ablation zone, wherein the second time is later than the first time, and overlaying, on the image, the second state of the progressive ablation zone. Element 9: further comprising receiving a third input prior to the amount of time elapsing and ceasing energizing the ablation probe based on the third input. Element 10: further comprising retrieving, from the memory, a dimension associated with the predicted ablation zone and overlaying, on the image, the dimension. Element 11: wherein the dimension comprises a longitudinal length of the predicted ablation zone, a lateral width of the predicted ablation zone, or a distance defined between a distal end of the ablation probe and a distal-most end of the predicted ablation zone, or combinations thereof. Element 12: wherein displaying an image representative of an ablation probe the image comprises displaying a three-dimensional image representative of the ablation probe. Element 13: wherein the display includes a time widget and a power widget, and wherein receiving the first and second inputs comprises receiving the first and second inputs via the time and power widgets, respectively. Element 14: further comprising receiving, from a temperature sensor, a temperature measurement and adjusting, on the display, the size of the progressive ablation zone based on the temperature measurement. Element 15: wherein to dynamically overlay a progressive ablation zone, the processor is to retrieve, from the memory at a first time, a first state of the ablation zone smaller than the predicted ablation zone, overlay, on the image, the first state of the progressive ablation zone, retrieve, from the memory at a second time, a second state of the ablation zone larger than the first state of the progressive ablation zone and smaller than the predicted ablation zone, wherein the second time is later than the first time, and overlay, on the image, the second state of the progressive ablation zone. Element 16: further storing instructions that, when executed by the processor, cause the processor to receive a third input prior to the amount of time elapsing and cease energizing the ablation probe based on the third input. Element 17: further storing instructions that, when executed by the processor, cause the processor to retrieve, from the memory, a dimension associated with the predicted ablation zone and overlay, on the image, the dimension.

By way of non-limiting example, exemplary combinations applicable to A, B, and C include: Element 1 with Element 2; Element 1 with Element 3; Element 1 with Element 3; Element 1 with Elements 3 and 4; Element 1 with Element 5; Element 1 with Element 6; Element 1 with Element 7; Element 2 with Element 3; Element 2 with Elements 3 and 4; Element 2 with Element 5; Element 2 with Element 6; Element 2 with Element 7; Element 3 with Element 4; Element 3 with Element 5; Element 3 with Element 6; Element 3 with Element 7; Elements 3 and 4 with Element 5; Elements 3 and 4 with Element 6; Elements 3 and 4 with Element 7; Element 5 with Element 6; Element 5 with Element 7; Element 6 with Element 7; Element 1 with at least two of Elements 2-7; Element 2 with at least two of Elements 1 and 3-7; Element 3 with at least two of Elements 1, 2, and 4-7; Element 4 with at least two of Elements 1-3 and 5-7; Element 5 with at least two of Elements 1-4, 6, and 7; Element 6 with at least two of Elements 1-5 and 7; Element 7 with at least two of Elements 1-6; Element 8 with Element 9; Element 8 with Element 10; Element 8 with Element 10; Element 8 with Elements 10 and 11; Element 8 with Element 12; Element 8 with Element 13; Element 8 with Element 14; Element 9 with Element 10; Element 9 with Elements 10 and 11; Element 9 with Element 12; Element 9 with Element 13; Element 9 with Element 14;Element 10 with Element 11; Element 10 with Element 12; Element 10 with Element 13; Element 10 with Element 14; Elements 10 and 11 with Element 12; Elements 10 and 11 with Element 13; Elements 10 and 11 with Element 14; Element 12 with Element 13; Element 12 with Element 14; Element 13 with Element 14; Element 8 with at least two of Elements 9-14; Element 9 with at least two of Elements 8 and 10-14; Element 10 with at least two of Elements 8, 9, and 11-14; Element 11 with at least two of Elements 8-10 and 12-14; Element 12 with at least two of Elements 8-11, 13, and 14; Element 13 with at least two of Elements 8-12 and 14; Element 14 with at least two of Elements 8-13; Element 15 with Element 16; Element 15 with Elements 16 and 17; and Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an ablation probe that includes an antenna;
a display; and
a controller in communication with the ablation probe and the display, the controller comprising a memory and being operable to:
receive first and second inputs indicative of an amount of time and a power level, respectively, to energize the ablation probe;
retrieve, from the memory, a predicted ablation zone corresponding to the first and second inputs;
display, on the display, an image representative of the ablation probe, and overlay, on the image, the predicted ablation zone;
energize the ablation probe at the power level; and
dynamically overlay, on the image, a progressive ablation zone based on the ablation probe being energized, wherein a size of the progressive ablation zone is retrieved from the memory based on the amount of time the ablation probe has been energized at the power level.

2. The system of claim 1, wherein to dynamically overlay a progressive ablation zone, the controller is operable to:
retrieve, from the memory at a first time, a first state of the ablation zone smaller than the predicted ablation zone;
overlay, on the image, the first state of the progressive ablation zone;
retrieve, from the memory at a second time, a second state of the ablation zone larger than the first state of the progressive ablation zone and smaller than the predicted ablation zone, wherein the second time is later than the first time; and
overlay, on the image, the second state of the progressive ablation zone.

3. The system of claim 1, wherein the controller is further operable to:
receive a third input prior to the amount of time elapsing; and
cease energizing the ablation probe based on the third input.

4. The system of claim 1, wherein the controller is further operable to:
retrieve, from the memory, a dimension associated with the predicted ablation zone; and
overlay, on the image, the dimension.

5. The system of claim 4, wherein the dimension comprises:
a longitudinal length of the predicted ablation zone;
a lateral width of the predicted ablation zone; or
a distance defined between a distal end of the ablation probe and a distal-most end of the predicted ablation zone; or
combinations thereof.

6. The system of claim 1, wherein the image comprises a three-dimensional image.

7. The system of claim 1, wherein the display includes a time widget and a power widget, and wherein the controller is further operable to receive the first and second inputs via the time and power widgets, respectively.

8. The system of claim 1, wherein the controller is further operable to:
receive, from a temperature sensor, a temperature measurement; and
adjust, on the display, the size of the progressive ablation zone based on the temperature measurement.

9. A method, comprising:
displaying, on a display, an image representative of an ablation probe;
receiving first and second inputs indicative of an amount of time and a power level, respectively, to energize the ablation probe;
retrieve, from the memory, a predicted ablation zone corresponding to the first and second inputs;
overlaying, on the image, the predicted ablation zone; and
dynamically overlaying, on the image, a progressive ablation zone based on the ablation probe being energized, wherein a size of the progressive ablation zone is retrieved from the memory based on the amount of time the ablation probe has been energized at the power level.

10. The method of claim 9, wherein dynamically overlaying a progressive ablation zone comprises:
retrieving, from the memory at a first time, a first state of the ablation zone smaller than the predicted ablation zone;
overlaying, on the image, the first state of the progressive ablation zone;
retrieving, from the memory at a second time, a second state of the ablation zone larger than the first state of the progressive ablation zone and smaller than the predicted ablation zone, wherein the second time is later than the first time; and
overlaying, on the image, the second state of the progressive ablation zone.

11. The method of claim 9, further comprising:
receiving a third input prior to the amount of time elapsing; and
ceasing energizing the ablation probe based on the third input.

12. The method of claim 9, further comprising:
retrieving, from the memory, a dimension associated with the predicted ablation zone; and
overlaying, on the image, the dimension.

13. The method of claim 12, wherein the dimension comprises:
a longitudinal length of the predicted ablation zone;
a lateral width of the predicted ablation zone; or
a distance defined between a distal end of the ablation probe and a distal-most end of the predicted ablation zone; or
combinations thereof.

14. The method of claim 9, wherein displaying an image representative of an ablation probe the image comprises displaying a three-dimensional image representative of the ablation probe.

15. The method of claim 9, wherein the display includes a time widget and a power widget, and wherein receiving the first and second inputs comprises receiving the first and second inputs via the time and power widgets, respectively.

16. The method of claim 9, further comprising:
receiving, from a temperature sensor, a temperature measurement; and
adjusting, on the display, the size of the progressive ablation zone based on the temperature measurement.

17. A non-transitory computer readable medium storing instructions that, when executed by a processor, cause the processor to:
receive first and second inputs indicative of an amount of time and a power level, respectively, to energize an antenna of an ablation probe;
retrieve, from a memory, a predicted ablation zone corresponding to the first and second inputs;
display, on a display, an image representative of the ablation probe, and overlay, on the image, the predicted ablation zone;
energize the ablation probe at the power level; and
dynamically overlay, on the image, a progressive ablation zone based on the ablation probe being energized, wherein a size of the progressive ablation zone is retrieved from the memory based on the amount of time the ablation probe has been energized at the power level.

18. The non-transitory computer readable medium of claim 17, wherein to dynamically overlay a progressive ablation zone, the processor is to:
retrieve, from the memory at a first time, a first state of the ablation zone smaller than the predicted ablation zone;
overlay, on the image, the first state of the progressive ablation zone;
retrieve, from the memory at a second time, a second state of the ablation zone larger than the first state of the progressive ablation zone and smaller than the predicted ablation zone, wherein the second time is later than the first time; and
overlay, on the image, the second state of the progressive ablation zone.

19. The non-transitory computer readable medium of claim 17, further storing instructions that, when executed by the processor, cause the processor to:
receive a third input prior to the amount of time elapsing; and
cease energizing the ablation probe based on the third input.

20. The non-transitory computer readable medium of claim 17, further storing instructions that, when executed by the processor, cause the processor to:
retrieve, from the memory, a dimension associated with the predicted ablation zone; and
overlay, on the image, the dimension.
